(19) United States
(12) Patent Application Publication
(10) Pub. No.: US 2005/0220710 A1
(43) Pub. Date: Oct. 6, 2005

(54) Overfeeding methods for determining therapeutic strategies and/or targets for obesity therapeutics

(76) Inventor: Randy S. Seeley, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 10/506,935

(22) PCT Filed: Mar. 7, 2003
(86) PCT No.: PCT/US03/07253

§ 371 Date: Apr. 25, 2005

Related U.S. Application Data
(60) Provisional application No. 60/362,813, filed on Mar. 8, 2002.

(51) Int. Cl. A61K 49/00; C12Q 1/68; G06F 19/00; G01N 33/48; G01N 33/50

(52) U.S. Cl. 424/9.2; 435/6; 702/20

(57) ABSTRACT

Methods for determining therapeutic strategies and/or targets for examining weight loss in an individual, comprising administering to the individual a prolonged overfeeding regimen and identifying gene and/or protein expression that occur in the individual with the prolonged overfeeding regimen.

### FIELD OF THE INVENTION

The present invention is directed toward methods for determining therapeutic strategies and/or targets for obesity therapeutics. More particularly, the methods are directed towards determining therapeutic strategies and/or targets for examining weight loss in an individual. The methods comprise administering to the individual a prolonged overfeeding regimen and identifying gene and/or protein expression that occur in the individual with the prolonged overfeeding regimen.

### BACKGROUND OF THE INVENTION

The growing prevalence of obesity in our society continues to be one of the most daunting and expensive public health problems. Considerable research effort has been aimed at understanding the physiological factors that produce caloric intake in excess of caloric need. Obesity, however, rather than resulting solely from taking in a caloric surplus, also reflects a failure in certain individuals to compensate for the consequent caloric surplus the way lean individuals do. That is to say, while lean individuals often go through periods where caloric intake exceeds metabolic needs, their food intake is subsequently reduced until body weight returns to baseline. In contrast, many individuals gain weight over the holiday period and they do not necessarily lose that weight during the post-holiday period.

While the regulatory responses to positive energy balance can be considered important in the genesis of obesity, they are also important targets for therapeutic intervention in already obese individuals. The need for effective treatments for individuals suffering from excessive body fat continues to rise as the rates of obesity in developed nations increase at an alarming rate. The normal response to positive energy balance involves the recruitment of endogenous systems that reduce food intake, increase energy expenditure and cause weight loss.

There is also a need for effective treatments for individuals suffering from wasting disorders. One possibility for the dramatic hypophagia and weight loss that accompanies illnesses such as AIDS and some cancers is the inappropriate activation of the same regulatory responses that suppress food intake after overfeeding. If the same systems are involved in cachexia, it would suggest that strategies aimed at antagonizing these response systems may be effective in promoting weight gain and increase both the life expectancy and the quality of life of patients suffering from these disorders. Finding potential ways to mimic or trigger these endogenous regulatory response systems could provide unique insights and therapeutic strategies for the treatment of obesity and wasting disorders.

Thus, there is a substantial need for methods of developing targets for obesity therapeutics or wasting disorders, and effective treatments for individuals suffering from obesity or wasting disorders.

### SUMMARY OF THE INVENTION

Accordingly, it is an object of this invention to provide methods of determining therapeutic strategies and/or targets for examining weight loss in an individual.

In accordance with one aspect of the invention, there are provided methods of determining therapeutic strategies and/or targets for examining weight loss in an individual. The methods comprise administering to the individual a prolonged overfeeding regimen and identifying gene and/or protein expression that occur in the individual with the prolonged overfeeding regimen.

In accordance with another aspect of the invention, there are provided methods of determining therapeutic strategies and/or targets for examining wasting disorders in an individual. The methods comprise administering to the individual a prolonged overfeeding regimen and identifying gene and/or protein expression that occur in the individual with the prolonged overfeeding regimen.

The present methods are advantageous for determining therapeutic strategies and/or targets to investigate weight loss or wasting disorder in an individual. Additional embodiments, objects and advantages of the invention will become more fully apparent in view of the following detailed description.

### DETAILED DESCRIPTION

Body weight (or more accurately, body adiposity) is a carefully controlled and regulated variable. Under normal conditions, individuals, when given ad lib access to food, precisely match caloric intake to caloric expenditure resulting in stable levels of stored calories in the form of adipose tissue. One example of this regulation is the robust regulatory response activated when individuals are put into a state of positive energy balance by being force-fed calories in excess of their caloric expenditure. During such a regimen, individuals dramatically suppress their spontaneous food intake while they gain weight and increase their body fat stores. This anorexigenic response during the overfeeding regimen is not surprising. The caloric solutions that are placed into the gastrointestinal tract activate a number of signals with demonstrated ability to suppress food intake, including gastric distension, release of cholecystokinin and bombesin-like peptides, and increase metabolic fuels in the circulatory system and liver.

In addition to suppressing food intake during the period in which calories are being infused, individuals maintain their low spontaneous food intake for several days after the overfeeding regimen is discontinued. This hypophagia results in loss of body weight and generally continues until body weight has returned precisely to the level of control individuals that were infused with equal volumes of a non-caloric solution. While signals related to the caloric infusions themselves may be responsible for the suppression of food intake during the overfeeding regimen, the candidate signals to mediate the hypophagia and body weight loss that occur after the overfeeding regimen are more limited: While not intending to be bound by theory, it is believed that an overfeeding regimen produces an increase in adipose tissue and this is associated with an elevation of hormonal negative feedback signals, generated by hormones including, but not limited to, insulin and leptin, that act in specific hypothalamic nuclei to alter biosynthesis and release of neuropeptide effector systems capable of changing both food intake and caloric expenditure.

In developing the present invention, the inventor recognizes that body weight (adiposity) is a regulated parameter, and understanding the physiology of the regulatory process forms the underpinning to treat obesity. The inventor has determined that when individuals are force-fed calories in excess of their caloric need (involuntary overfeeding), their spontaneous food intake drops to near zero and they gain body weight. Additionally, for some time after the termination of the overfeeding regimen, spontaneous food intake remains low until body weight has returned to control levels. Given that appropriate responses to both negative and positive energy balance are important for the accurate matching of caloric intake and caloric expenditure, the systems that mediate the anorectic response to overfeeding are believed to be sites involved in the genesis of obesity and also targets for treatment. Therefore, the inventor has determined that the response to overfeeding, the reciprocal limb of body weight regulation, is important to understanding and treating obesity.

In accordance, the present inventor has developed methods of determining therapeutic strategies and/or targets for examining weight loss in an individual. The methods comprise administering to the individual a prolonged overfeeding regimen and identifying gene and/or protein expression that occur in the individual with the prolonged overfeeding regimen. In addition, methods in accordance with the present invention may be used to determine therapeutic strategies and/or targets for examining wasting disorders in an individual. As used herein, “individual”, is intended to refer to an animal, including but not limited to humans, mammals, or rodents.

In one embodiment of the invention, the prolonged overfeeding regimen is administered at least 3 days. In another embodiment of the invention, the prolonged overfeeding regimen is administered at least 5 days. In yet another embodiment of the invention, the prolonged overfeeding regimen is administered at least 1 week. In a further embodiment of the invention, the prolonged overfeeding regimen is administered at least 2 weeks. In a further embodiment of the invention, the prolonged overfeeding regimen is administered at least 4 weeks. In a further embodiment of the invention, the prolonged overfeeding regimen is administered at least 6 weeks. The upper and/or lower limit of days/weeks necessary for the prolonged overfeeding regimen may vary in individual embodiments of the present method, and in specific embodiments may be 3 days, 5 days, 1 week, 2 weeks, 4 weeks, or 6 weeks, or the like.

Anything that provides calories to the individual may be used for the prolonged feeding regimen. In one embodiment of the invention, the calories are derived from Osmolite, as shown in Example 1. Furthermore, one skilled in the art will recognize the various methods that may be employed for an individual to receive the calories of the prolonged feeding regimen. In one embodiment of the invention, the individual receives calories directly into the stomach during the administration of the prolonged overfeeding regimen. In a further embodiment of the invention, the prolonged overfeeding regimen is administered though a gastric catheter.

Levels of gene and/or protein expression can be measured at any time during the method. In one embodiment of the invention, the gene and/or protein expression is identified during or after the prolonged overfeeding regimen. In another embodiment, a referenced gene and/or protein expression is identified before the prolonged overfeeding regimen.

In accordance with one embodiment of the invention, the step for identifying gene and/or protein expression that occur in an individual with the prolonged overfeeding regimen comprise collecting a tissue sample from the individual, isolating RNA, DNA, protein or combinations thereof from the tissue, determining a level of expression of a the RNA, DNA, protein or combinations thereof, analyzing the level of expression, and defining a target based on the analysis.

In a further embodiment, the gene and/or protein expression is identified in a tissue sample. The tissue samples include, but are not limited to, brain tissue, stomach tissue, intestine tissue, white fat tissue, brown fat tissue, muscle tissue, pancreas tissue, pituitary tissue, liver tissue, bone tissue or combinations thereof. The brain tissue includes, but is not limited to, hypothalamus tissue, brainstem tissue, midbrain tissue, forebrain tissue or combinations thereof.

Gene and/or protein expression derived from the tissue collected may be derived from, but are not limited to, specific hormonal and neuropeptide systems that mediate the potent regulatory response to involuntary overfeeding and that serve to restore energy balance. More specifically, while not wishing to be bound by theory, it is believed that the Central Nervous System (CNS) melanocortin signaling role is important for inhibiting food intake in the normal regulation of energy balance. This is due to the present inventor having determined that individuals who are involuntarily overfed have elevated expression of the melanocortin precursor, proopiomelanocortin (POMC) in the arcuate nucleus (ARC) of the hypothalamus. Consistent with elevated POMC activity, the potent hypophagic response that follows a period of involuntary overfeeding is completely blocked by central administration of a melanocortin receptor antagonist at doses that have no effect on food intake in non-overfed individuals.

In addition to these systems, gene and/or protein expression may be derived from specific molecules, which may be involved in the cascade of events that involves genomic and/or proteomic effects that result in an individual biased to consume more food. As used herein, “molecules” is intended to refer to genes, proteins or combinations thereof. For example, the molecules, which create the gene and/or protein expression associated with the prolonged overfeeding regimen may include, but are not limited to, agouti-related protein (AgRP), proopiomelanocortin (POMC), α-melanocyte stimulating hormone (α-MSH), and melanocortin receptors, such as MC3 and MC4.

Specifically, the antagonist AgRP produces a potent and sustained state of positive energy balance when administered into the 3^{rd} cerebral ventricle (i3vt). AgRP causes elevated food intake and weight gain for up to 6 days as a result of a single injection. Moreover, while not wishing to be bound by theory, the inventor has further determined that the mechanism for this sustained orexigenic effect is not the result of continued melanocortin receptor antagonism, but rather is believed to result from circuits mediating unique short and long-term effects of AgRP on food intake.

Furthermore, the precursor molecule of hypothalamic melanocortins is POMC. In addition to several other important neuropeptides, POMC encodes α-melanocyte stimulating hormone (α-MSH), a melanocortin that is an agonist at several melanocortin receptors. When given into the 3^{rd} ventricle, A-MSH and other non-selective melanocortin receptor agonists (including the synthetic analogue, MTII) reduce food intake and body weight, whereas administration of melanocortin receptor antagonists (such as SHU-9119) increases food intake and body weight. Furthermore, within the hypothalamus there are two identified melanocortin receptors, MC3 and MC4. The present inventor has determined that, when administered into the 3^{rd} ventricle of the rat, selective MC4 receptor agonists inhibit and selective MC4 antagonists stimulate food intake. Involuntary overfeeding increases POMC gene expression in the ARC and the hypophagia that follows a period of involuntary overfeeding can be blocked by central melanocortin receptor blockade. All of this evidence points to the endogenous POMC/α-MSH/MC4 hypothalamic system as being a key catabolic effector pathway capable of eliciting robust decreases of food intake and body weight.

In a further embodiment of the invention, the method for determining the level of expression may comprise preparing a probe using the isolated RNA, DNA, protein or combinations thereof, applying the probe to an array, and measuring the level of the RNA, DNA, protein or combinations thereof, of the array. In a further embodiment of the invention, the method for analyzing the level of expression comprises performing bioinformatic analysis. The bioinformatic analysis include, but are not limited to, statistical analysis, class prediction, clustering, computer programs, or combinations thereof.

In summary, over long intervals, caloric intake matches caloric expenditure. This matching requires that individuals respond to states of both negative and positive energy balance. While negative energy balance has been widely studied, the responses to positive energy balance have received far less attention. The systems and/or molecules involved in both the negative and positive energy balance are important to understanding the genomic and/or proteomic effects that result in an individual that is biased to consume more food. Therefore, the present methods are advantageous for determining therapeutic strategies and/or targets for examining weight loss and/or wasting disorders in an individual.

The foregoing description of the various embodiments of the invention has been presented for the purposes of illustration and description. It is not intended to be exhaustive or to limit the invention to the precise form disclosed. Many alternatives, modifications and variations will be apparent to those skilled in the art of the above teaching. Accordingly, this invention is intended to embrace all alternatives, modifications and variations that have been discussed herein, and others that fall within the spirit and broad scope of the claims.

### EXAMPLE

This example demonstrates the method for determining targets and/or strategies for obesity therapeutics. 72 young adult male Long Evans rats are prepared with surgically implanted gastric catheters. Gastric catheters are constructed from 15 cm of silastic tubing and a 1-cm dacron mesh disc. The mesh end of the catheter is inserted through the stomach wall and two small sutures close the incision around the tubing. The catheter is then brought through the muscle wall and run subcutaneously to the incision over the skull. A short length of 23-g metal tubing is force-fit into the silastic tubing of the catheter. Screws are placed in the parietal plates of the skull and the cannula is secured to the screws with dental acrylic.

The 72 rats are divided into 3 groups. 30 of the rats are overfed for a period of 20 days via the gastric catheter. Meals are delivered into the stomach via a gastric catheter and a multi-head peristaltic pump. Over days 1-20, gastric infusions are increased incrementally from one meal of 10 ml on the first day to five meals of 15 ml of Osmolite (Ross Products Division Abbott Laboratories, Columbus, Ohio) at a rate of 1 ml/min. After completion of the 20-day overfeeding regimen, rats are given free access to pelleted chow. Groups of 3 rats are sacrificed every 12 hours for the next 5 days for a total of 10 time points. At sacrifice, each rat has brain (forebrain, hypothalamus and brainstem), epididymal and retroperitoneal adipose tissue, 3 segments of GI tract, soleus muscle, liver and pancreas taken for later expression profiling.

Another group of 12 rats are given gastric infusions of non-caloric, isotonic saline in volumes identical to that of the overfed group. After 20 days of this regimen individuals are sacrificed 12 hours apart on day 1 and again on day 5 (n 3/timepoint). This group is used to compare the gene changes that occur over a 5-day period in this experiment in rats that have not had any perturbation in their energy balance. Such saline infusions do not alter food intake or weight gain in rats.

The final group of 30 rats provides the most important comparison for the overfed rats. Like the previous group, they receive volume-matched infusions of saline for a 20-day period. However, rather than having free access to chow after the 20-day intervention, they are allowed to consume only what the overfed group spontaneously consumed. In this way, this “pairfed” group is consuming exactly the same number of calories as the overfed group. The salient difference is that the overfed group actively chooses to consume that number of calories while the pairfed group would consume more if it were allowed. Such a group increases the probability of finding genes that are part of the response to positive energy balance and mediate the reduction in hunger rather than genes that are simply responding to differing nutrient loads. Like the overfed group, individuals in this group are sacrificed every 12 hours for the next 5 days (n=3/group) for tissue collection.

The tissue samples are placed in approximately 20 ml of RNAlater (Ambion) and stored overnight at 4° C. The RNAlater is poured off the next day and the tissue stored at −80° C. Total RNA is extracted from the tissue using RNA Stat-60 (Tel-Test Inc.), according to the manufacturer's guidelines. The total RNA is then cleaned up by passage through a Qiagen mini-column and the amount of RNA within the sample is determined. In addition, proteins are also separated and stored for later analysis.

After the isolation of the total RNA, twenty (20) μg of total RNA is provided to a member of the gene expression core facility, and they use 10 μg of the sample to label targets. The RNA samples are labeled, hybridized to a commercially available GeneChip (Affymetrix). The level of expression of the gene and/or protein isolated are determined and analyzed by bioinformatics analyses. This method demonstrates how involuntary overfeeding develops strategies and/or targets for obesity therapeutics.

The specific embodiment and example set forth above is provided for illustrative purposes only and are not intended to limit the scope of the following claims. Additional embodiments of the invention and advantages provided thereby will be apparent to one of ordinary skill in the art and are within the scope of the claims.

1. A method of determining therapeutic strategies and/or targets for examining weight loss in an individual, comprising administering to the individual a prolonged overfeeding regimen and identifying gene and/or protein expression that occur in the individual with the prolonged overfeeding regimen.

2. A method according to claim 1, wherein the individual receives calories directly into the stomach during administration of the prolonged overfeeding regimen.

3. A method according to claim 2, wherein the prolonged overfeeding regimen is administered through a gastric catheter.

4. A method according to claim 1, wherein the gene and/or protein expression is identified during or after the prolonged overfeeding regimen.

5. A method according to claim 4, wherein a referenced gene and/or protein expression is identified before the prolonged overfeeding regimen.

6. A method according to claim 1, wherein the gene and/or protein expression is identified in a tissue sample.

7. A method according to claim 6, wherein the tissue sample comprises brain tissue, stomach tissue, intestine tissue, white fat tissue, brown fat tissue, muscle tissue, pancreas tissue, pituitary tissue, liver tissue, bone tissue or combinations thereof.

8. A method according to claim 7, wherein the brain tissue comprises hypothalamus tissue, brainstem tissue, midbrain tissue, forebrain tissue or combinations thereof.

9. A method according to claim 1, wherein identifying gene and/or protein expression that occur in an individual with the prolonged overfeeding regimen comprises:
a. collecting a tissue sample from the individual;
b. isolating RNA, DNA, or protein or combinations thereof from the tissue;
c. determining a level of expression of the isolated RNA, DNA, protein or combinations thereof;
d. analyzing the level of expression; and
e. defining a target based on the analysis of the level of expression.

10. A method according to claim 9, wherein determining the level of expression comprises:
a. preparing a probe using the isolated RNA, DNA, protein or combinations thereof,
b. applying the probe to an array, and
c. measuring the level of the RNA, DNA, protein, or combinations thereof, of the array.

11. A method according to claim 9, wherein analyzing the level of expression comprises performing bioinformatic analysis.

12. A method according to claim 11, wherein bioinformatic analysis comprises statistical analysis, class prediction, clustering, computer programs, or combinations thereof.

13. A method of determining therapeutic strategies and/or targets for wasting disorders in an individual, comprising administering to the individual a prolonged overfeeding regimen and identifying gene and/or protein expression that occur in the individual with the prolonged overfeeding regimen.
